(19) United States
(12) Patent Application Publication
(10) Pub. No.: US 2014/0052109 A1
(43) Pub. Date: Feb. 20, 2014

(54) Steerable Multifunction Catheter Probe with High Guidability and Reversible Rigidity

(71) Applicant: Diros Technology Inc., Markham, ON (US)

(72) Inventors: Leslie William Organ, Charleston, SC (US); Peter George Darmos, Willowdale (CA); Joel Ironstone, Toronto (CA); George Peter Darmos, Willowdale (CA); Ilya Gavrilov, Mississauga (CA)

(73) Assignee: Diros Technology Inc., Markham, ON (unknown)

(21) Appl. No.: 13/970,590

(22) Filed: Aug. 19, 2013

Related U.S. Application Data
(60) Provisional application No. 61/684,779, filed on Aug. 19, 2012.

(51) Int. Cl. A61M 25/01

(52) U.S. Cl. CPC A61M 25/0147
USPC 604/528

(57) ABSTRACT

A steerable, multifunction radiofrequency (RF) catheter probe for providing improved steerability plus variable rigidity of RF catheter probes. The features of the invention include the ability to reversibly vary the rigidity of catheter probes and to configure the shape of the distal end of catheter probes as required by the application.

### CROSS REFERENCE TO RELATED APPLICATIONS

This application claims the benefit of U.S. Provisional Application No. 61/684,779, filed Aug. 19, 2012, which is incorporated herein by reference in its entirety.

### BACKGROUND OF THE INVENTION

1. Field of the Invention

The present invention relates to the field of catheter probes and, more specifically, to steerable multifunction catheter probes for diagnostic and/or therapeutic purposes.

2. Description of the Related Art

The insertion of catheters and probes into one of an epidural space, a spinal space, or a paravertebral space of a patient to treat chronic neurogenic pain not relieved by more conservative medical procedures is well known. For example, epidural catheters can be inserted into the epidural space and, by fluoroscopic and/or endoscopic guidance, reach a target area at which point local anesthetics and steroids can be injected to relieve the pain. The catheter can remain in place for one to 30 days, for example, and the injection of the medications can be made through external or implanted pumps.

Alternatively or in addition to the above treatment, a probe, inserted in combination with or sequential to the catheter, can be used to apply continuous or pulsed radiofrequency (RF) energy as a therapeutic modality to at least one of a nerve, a nerve root, a nerve ganglion, or a part of the spinal cord. Also, low frequency electrical stimulation can be used to assist with the identification of target structures prior to treatment with steroids or RF energy, or to assess the effectiveness of treatment by comparing sensory responses, for example in the lower limbs, before and after treatment. Thus, the use of catheters and probes in epidural, spinal, and paravertebral spaces to treat chronic neurogenic pain is generally accepted, but is limited because conventional catheters and probes can lack tip directionality or variable probe rigidity for the guidability needed to access some regions for diagnostic and treatment procedures. In addition, such catheters and probes, or combined catheter probes, have application and uses in other body regions not described in the following disclosures.

### BRIEF SUMMARY OF THE INVENTION

To achieve the foregoing and other objects, the present invention, as embodied and broadly described herein, provides various embodiments of steerable multifunction catheter probes with high guidability and reversible rigidity for diagnostic and/or therapeutic purposes

The present invention is a steerable multifunction catheter probe with high guidability and reversible rigidity for diagnostic and/or therapeutic purposes. In an embodiment, the catheter probe includes a catheter body having a body portion adapted for being connected to a proximal hub and a distal end portion connected to the body portion, wherein the catheter body defines a lumen; the distal end portion having a compressible segment and a non-compressible segment, the compressible segment having a longitudinal centerline; and a pull member attached to the distal end portion and adapted for applying a proximally directed force to the distal end portion whereby at least a portion of said compressible segment is compressed.

In other embodiment the catheter probe includes a catheter body having a body portion adapted for being connected to a proximal hub and a distal end portion connected to the body portion, wherein the catheter body defines a lumen; the distal end portion having a first compressible segment, second compressible segment disposed distally from the first compressible segment, and a non-compressible segment disposed between the first and second compressible segments, the compressible segments each having a longitudinal centerline; and a first pull member attached to the distal end portion and adapted for applying a proximally directed force to the distal end portion whereby at least a portion of said first compressible segment is compressed.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment. The skilled person in the art will understand that the drawings described below are for illustration purposes only and are not intended to limit the scope of the applicants' teachings in any way.

FIG. 1 shows an example embodiment of a distal end portion of a steerable catheter probe;

FIG. 2 shows flexion of the distal end portion of the steerable catheter probe of FIG. 1;

FIGS. 3A-C are used for the description of the factors contributing to the flexibility of the catheter probe distal end portion;

FIGS. 4A-E show example embodiments of construction of the distal end portion of catheter probes based on the factors described in FIG. 3;

FIG. 5 is an example embodiment of a steerable catheter probe with two pull wires;

FIGS. 6A-E show examples of coils with reduced cross sectional areas and variable shapes within the distal end portion of steerable catheter probes that can be used as alternative constructions for enabling multidirectional tip deflection;

FIGS. 7A-D relate to examples of catheter probes in which the distal end portion (and intermediate tubular body, not shown) can be made reversibly rigid;

FIGS. 8A-B show an example embodiment of handle device for pulling a pull member attached to the distal end portion and thereby applying a proximally directed force thereto whereby at least a portion of a compressible coil is compressed causing flexion of the distal end portion; and

FIGS. 9A-C show an example embodiment of handle device for pulling a plurality of pull members attached to the distal end portion, whereby the pull members may operably function individually or in combination to provide multidirectional flexion control of or rigidity to the distal end portion.

### DESCRIPTION OF THE PREFERRED EMBODIMENTS

Various apparatuses or processes will be described below to provide examples of embodiments of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses or processes described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors or owners do not intend to abandon, disclaim or dedicate to the public any such invention by its disclosure in this document.

It should be noted that the term “catheter probe” used herein is meant to represent a medical device that comprises at least some of the functionality of both a catheter and a probe. It should also be noted that the term “hub” used herein is meant to represent an element that can be used as a handle to hold the catheter probe as well as to provide electrical connections, fluid injectability, and the like. Furthermore, the term “distal” is used to generally indicate an element or portion of an element of a catheter probe that is located closer to the working end of the catheter probe and further away from the hub of the catheter probe. The term “proximal” is used to generally indicate an element or portion of an element that is located closer to the hub of the catheter probe and further away from the working end of the catheter probe. The term “working end” typically means the portion of the catheter probe that is first inserted into a patient and is also the portion of the catheter probe that provides various functions, such as at least one of fluid discharge, RF ablation, temperature sensing and the like.

The various embodiments described herein generally relate to steerable catheter probes that provide the functionality of catheters and probes for diagnostic and therapeutic purposes. They are steerable to facilitate, and in some cases make uniquely possible, access to various regions such as but not limited to an epidural space, a spinal space, or a paravertebral space for diagnostic and therapeutic procedures to treat chronic neurogenic pain not relieved by more conservative methods. The various steerable catheter probes to be described herein can also be used in other areas of a patient's body. Accordingly, the steerable catheter probes to be described herein may make possible an enlarged range of applications at a greater number of locations as compared to conventional catheters and probes. Furthermore, the various embodiments of the steerable catheter probes described herein may be supplied, if so desired, as a packaged, sterilized, single use disposable product or alternatively as a sterilizable, reusable product.

### Basic Flexion Mechanism of the Catheter Probe Distal End Portion

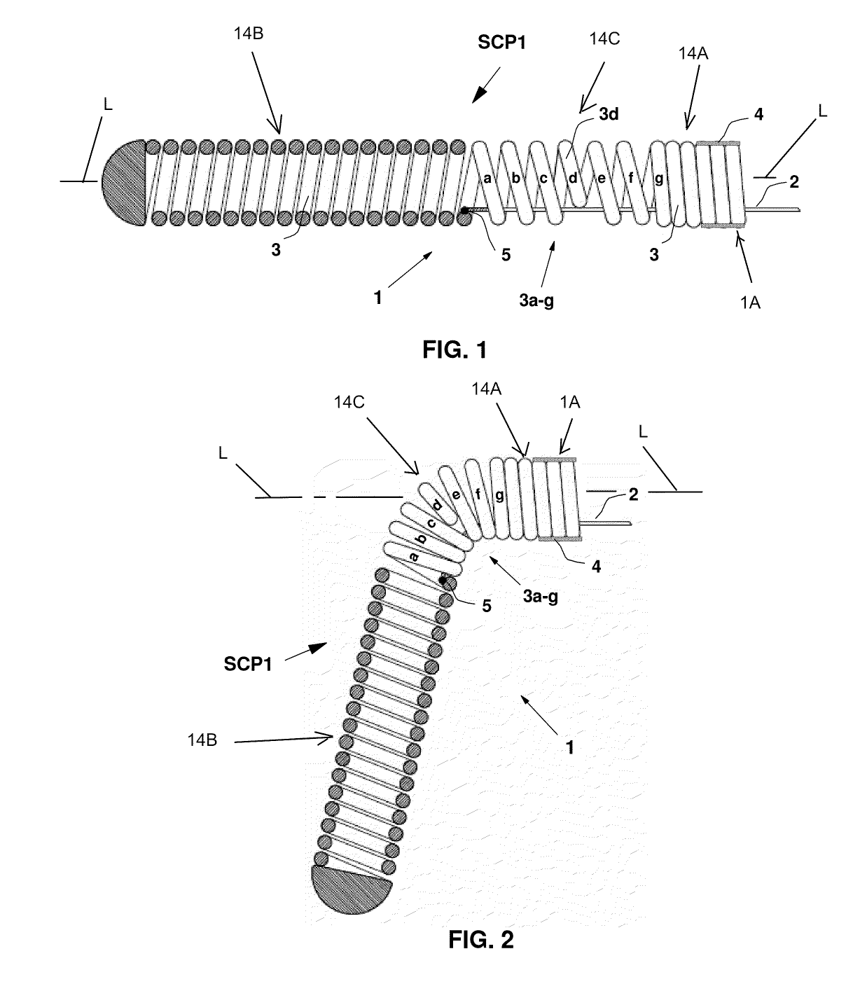

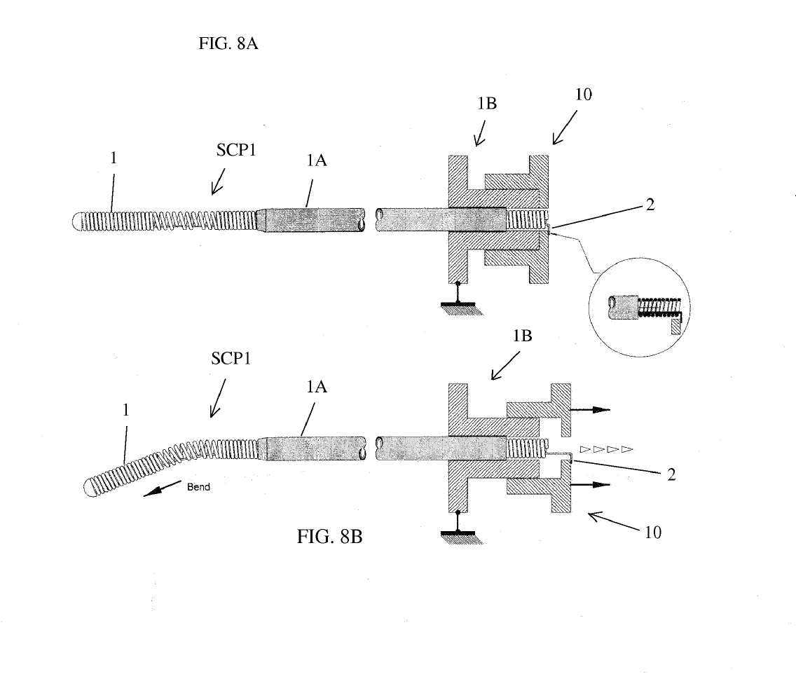

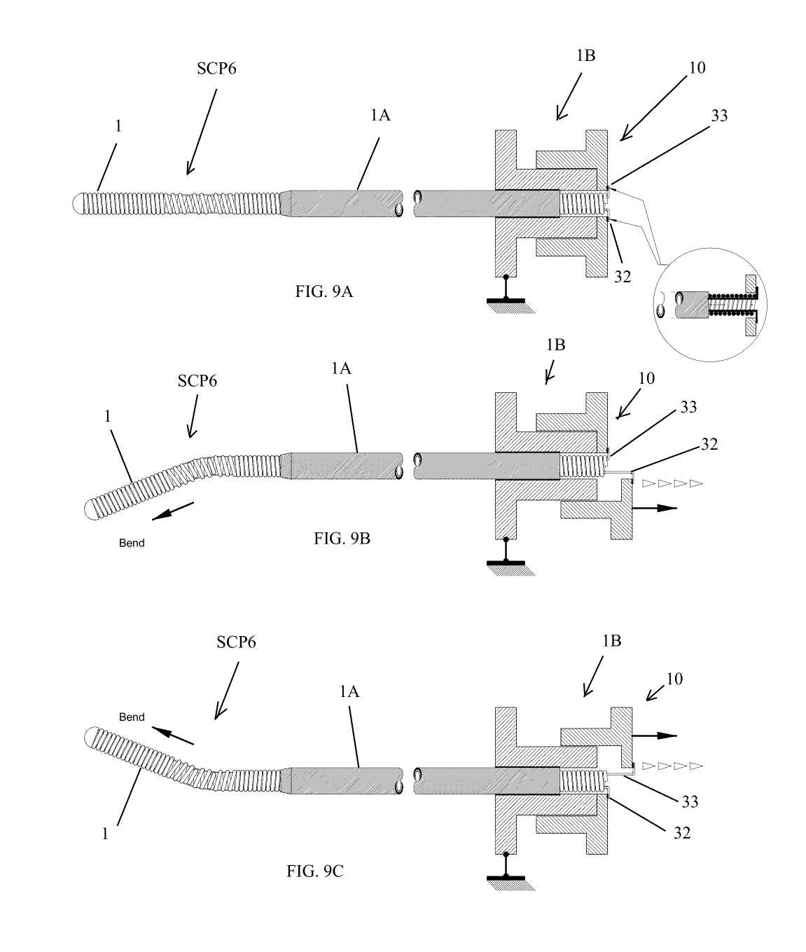

An example embodiment of a steerable catheter probe SCP1 of the present invention is shown in FIG. 1. Steerable catheter probe SCP1 comprises a tubular catheter body portion 1A and a catheter distal end portion 1, the tubular catheter body portion 1A being connected to a proximal hub member 1B as illustrated in FIGS. 8A-B. The proximal hub member 1B provides a handle or other actuation mechanism/device 10 which is operably coupled to a pull member 2 so as to cause the distal end portion 1 of the catheter probe SCP1 to deflect with respect to its longitudinal axis (L). Further, the handle or actuation mechanism is arranged so that it acts on at least one pull wire 2 in specific ways so as to cause the distal end portion 1 of steerable catheter probe SCP1 to be moved with respect to the longitudinal axis (L) of the catheter body portion 1A, in this example by the application of a proximally directed pulling force (F) on pull member 2, for example a wire, link or the like. FIGS. 8A-B shows an example embodiment of handle device 10 for pulling a pull member 2 attached to the distal end portion 1 and thereby applying a proximally directed force thereto whereby at least a portion of a compressible segment is compressed causing flexion of the distal end portion 1. FIGS. 9A-C show an example embodiment of handle device 10 for pulling a plurality of pull members 32, 33 attached to the distal end portion 1, whereby the pull members 32, 33 may operably function individually or in combination to provide multidirectional flexion control of or rigidity to the distal end portion 1.

The tubular catheter body portion 1A and at least one segment (14A and 14B illustrated) of the distal end portion 1 is operatively non-compressible along the longitudinal axis (L) when subject to an operational compressive load. The distal end portion 1 also includes at least one segment 14C that is operatively compressible along the longitudinal axis (L) when subject to an operational compressive load. In the preferred embodiment, the body portion 1A and distal end portion 1 comprise a continuous coil 3, wherein tightly wound adjacent coil loops are engaged thereby forming the non-compressive segments (body member 1A and segments 14A and 14B) and loosely wound adjacent coil loops 3a-g spaced apart (i.e. open) as a compression coil thereby forming the compressible segment 14C.

Part of the coil 3 is illustrated with the front half of the coil removed to show a full section view in order to expose a fixation point 5 for the pull wire 2. The tubular catheter body portion 1A is constructed of a tightly wound coil which continues to or partly comprises the catheter probe distal portion 1. The coil 3 is made of surgical grade stainless steel that has a smooth polymer coating 4 or other suitable insulator over the tubular catheter body portion and, variably, the proximal part of catheter probe distal end portion 14A. The catheter probe distal end portion 1 is otherwise generally uninsulated in its entirety, but may be partially insulated as determined by the application. It should be understood throughout this description that stainless steel is but one type of material that can be used to implement the coil 3 and that the coil 3 can also be made from titanium, nickel/titanium alloys (Nitinol) as well as various other medical grade metals as is known by those skilled in the art. The coil 3, i.e. the intermediate catheter body portion 1A and distal end portion 1 provide a housing for one or more electrically conductive pathways and/or an injection pathway. The generally tightly wound coil construction allows for the flexibility of steerable catheter probe SCP1 while maintaining a 1:1 torque capability for guidance control.

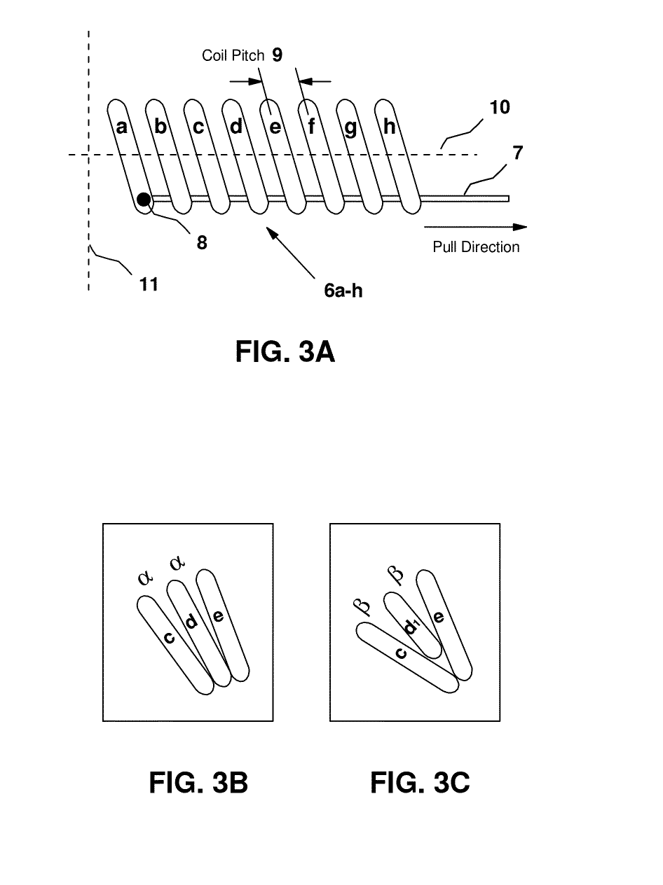

A feature of this invention is the enabling of a large range of flexion of catheter distal end portion 1 as illustrated in FIG. 2. It is based on the use of a compression coil configuration as the compressible segment 14C and, in this example, at least two different coil diameters in the segment 14C. An example embodiment is shown in FIGS. 1 and 2 comprising compression coil 3a-g with one of its members, coil 3d, of smaller diameter than the other larger and equal diameter coils. Pull wire 2 is attached at fixation point 5 to one of the large diameter coils distal to small diameter coil 3d. Pull wire 2 is within the lumen of the large diameter coils but outside the lumen of any smaller diameter coils. The consequence of this configuration is that when pull wire 2 is pulled proximally, deflection of the tip 18 occurs as shown in FIG. 2 with the catheter distal end portion 1 bent to a flexed position after the pull. Bending occurs because of the resultant angulation and compression of coils 3a-f, with the largest angulation between large diameter coils c and e on either side of smaller diameter coil 3d. The flexion angle can be substantial, and is limited only when the lower edges of large diameter coils 3c and 3e abut.

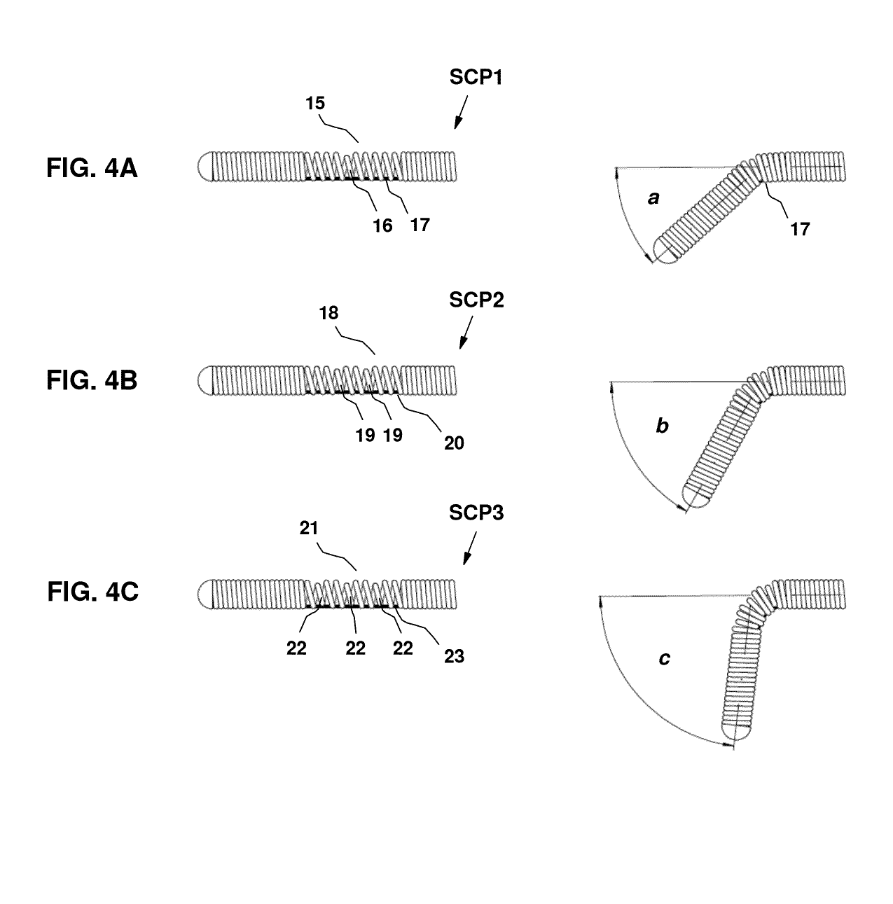

The factors contributing to the flexion of catheter distal end portion 1 are provided in more detail in FIGS. 3A-C. FIG. 3A shows a flexible segment compression coil 6a-h with equal diameter coils, a pull wire 7, its fixation point 8 to coil a, and the pull direction. Fixation point 8 may be to any other coil or structure distal to some or all the coils of the compression coil. As illustrated in FIG. 3A, coil pitch 9 is defined as the distance between adjacent coils of compression coil 6a-h. Also shown are the compression coil central longitudinal axis 10 and a reference y-axis 11. Generally for the various embodiments, the longitudinal axis L of the steerable catheter probe and central longitudinal axis of the distal end portion will be coaxial when the distal end portion is not in flexion.

In the preferred embodiment, there are three conditions to achieve the large flexion capabilities of the catheter distal end portion 1 of this invention:

A first condition is that coil pitch 9 is such that adjacent coils do not contact each other, i.e. the catheter distal end portion 1 of steerable catheter probe SCP1 is, at least in part, constructed as a compression coil.

A second condition is that in order for flexion of compression coil 6a-h to occur, the fixation point 8 and pull wire 7 must be above or below the coil central longitudinal axis L in order to create a bending moment in the positive or negative y-axis direction respectively when force (F) is applied in the indicated direction. In FIG. 3A the pull wire 7 is below coil central longitudinal axis L with the resultant deflection in the negative y-axis direction as shown for coils c, d, and e in FIG. 3B with angulation (α) degrees between coils c and d and d and e. Flexion increases with increased pull until there is engagement between the bottom edges of the coils.

It follows from the first and second conditions that, within limits, increasing compression coil pitch will increase distal end portion 1 flexibility.

An optional third condition, the one that provides maximum bending, is that at least one of the compression coils is smaller in diameter or cross sectional area than the others. For example, in the configuration of FIG. 3C where the diameter of coil d1 is less than that of coils c and e, application of a pulling force will now enable the bottom edges of coils c and e to come into contact, with resultant angles (β) being larger than angles (α) of adjacent coils of equal diameter.

### Example Embodiments of this Invention

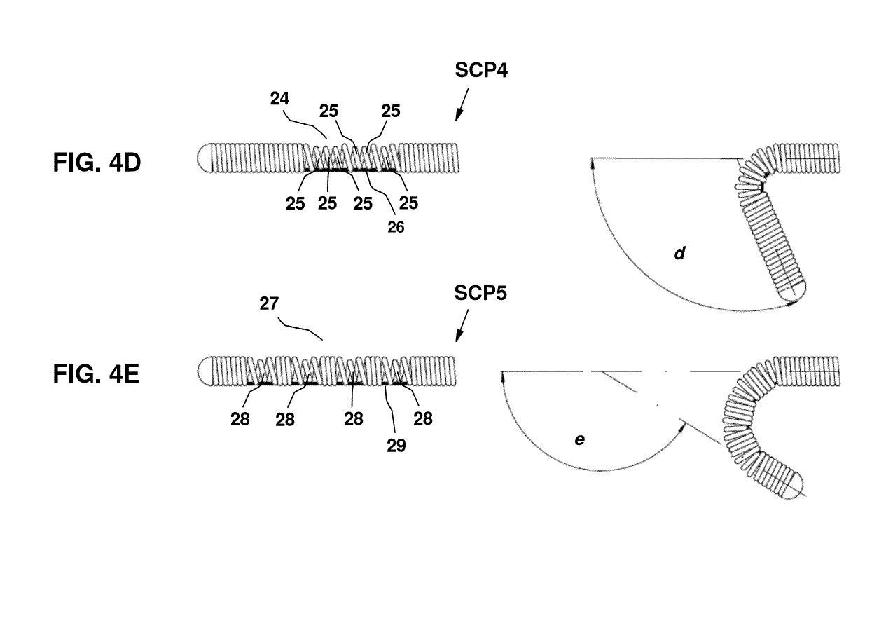

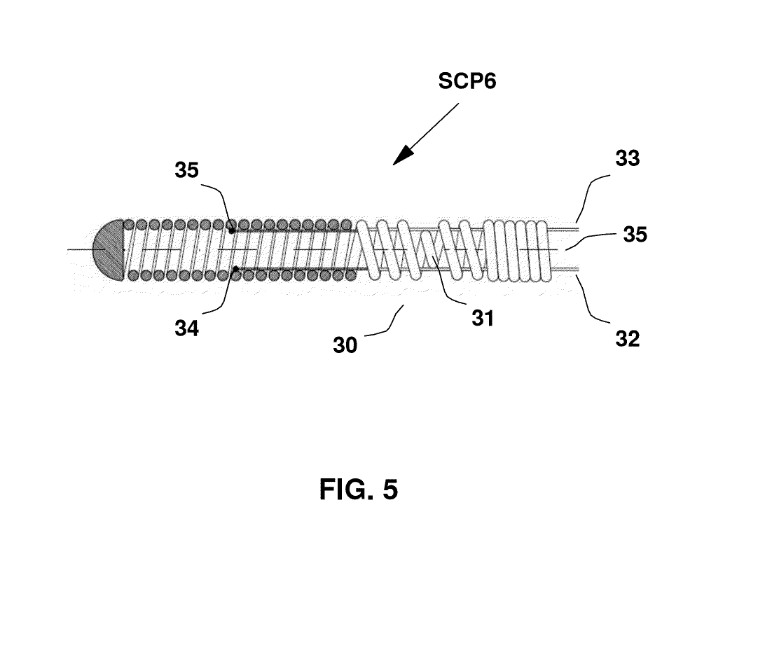

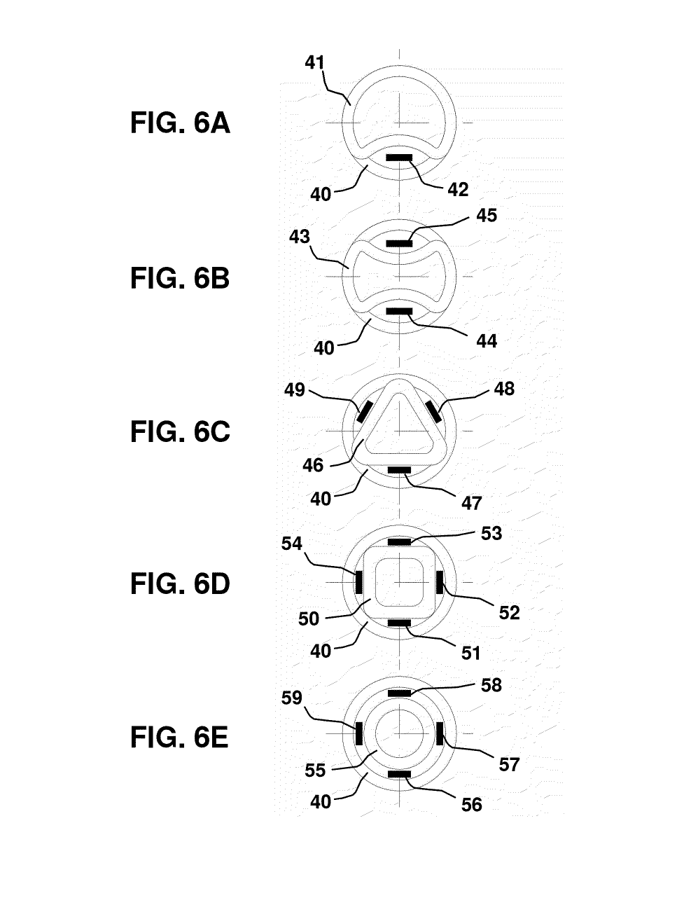

An example embodiment of this invention has been shown in FIGS. 1, 2 and 3C wherein catheter distal end portion 1 contains one coil of smaller diameter to enhance its flexibility. In alternative embodiments there can be at least one compression coil having: equal diameter coils (FIGS. 3A-B, 7A-B), multiple smaller diameter coils to further enhance flexibility and/or control the shape of the flexed catheter distal end portion according to particular requirements of use (FIGS. 4A-4E), multiple pull wires (FIGS. 5, 6B-6E, 7C, 7D) to control flexion and/or rigidity, and/or a smaller diameter coil having a non-circular cross-section (FIGS. 6B-6D). Each of these alternative embodiments has a proximal hub member 1B and tubular catheter probe body portion 1A as described herein in reference the embodiment of FIGS. 1, 2, 3C (these elements being incorporated by reference into the alternative embodiments), except that the actuation mechanism operates the number of pull wires corresponding to the particular embodiment.

FIG. 4A shows the distal end portion of a steerable catheter probe comprising in part a compression coil 15 that contains a single smaller diameter coil 16, the configuration of SCP1 in FIGS. 1 and 2. There is a pull wire 17 which when pulled proximally in the longitudinal axis of SCP1 will cause the distal end portion 1 of SCP1 to change from its relaxed, straight state of FIG. 4A left to the compressed, flexed state of a degrees as indicated in FIG. 4A right.

In an alternative embodiment shown in FIG. 4B, two smaller diameter coils 19 are incorporated into compression coil 18 of the catheter distal end portion of steerable catheter probe SCP2. In this, and other catheter probe distal ends with multiple smaller diameter coils, the smaller diameter coils do not need to be the same size, but must be smaller in diameter than other coils within the compression coil. With a proximal pull of pull wire 20 in the longitudinal axis of SCP2, the distal end portion 1 of SCP2 will change from its relaxed, straight state of FIG. 4B left to the compressed, flexed state of b degrees shown in FIG. 4B right, where the angle b is greater than the angle a of the single smaller diameter coil of FIG. 4A.

In another alternative embodiment, three smaller diameter coils 22 are incorporated into compression coil 21 of the catheter distal end portion of steerable catheter probe SCP3 as shown in FIG. 4C. When the pull wire 23 is pulled proximately in the longitudinal axis of SCP3, the distal end portion of SCP3 will change from its relaxed, straight state of FIG. 4C left to the compressed, flexed state of c degrees shown in FIG. C right, where the angle c is greater than the angle b of the distal end portion of SCP2 of FIG. 4B which contains two smaller diameter coils.

In yet another alternative embodiment, compression coil 24 of the catheter distal end portion of steerable catheter probe SCP4, shown in FIG. 4D, has three segments of smaller diameter coils 25, with one smaller diameter coil 25 in the most proximal segment, two smaller diameter coils 25 in the next segment, and three smaller diameter coils 25 in the most distal segment. When the pull wire 26 is pulled proximately in the longitudinal axis of SCP4, the distal end portion of SCP4 will change from its relaxed, straight state of FIG. 4D left to the compressed, flexed state of d degrees shown in FIG. 4D right, where the angle d is greater than the angle c of the distal end portion of SCP3 of FIG. 4C which contains three segments each with one smaller diameter coil only.

An additional benefit of uneven distribution and variable number of smaller diameter coils incorporated within a compressed coil is that by judicial selection of the position and/or number of smaller diameter coils, the curvature the distal end portion in the flexed state can be made to conform to a specified shape optimal for a desired use. This is demonstrated in the example embodiment of FIG. 4E which has four smaller diameter coils 28, one incorporated into each of four segments of compression coil 27 of catheter distal end portion 1 of steerable catheter probe SCP5. When the pull wire 29 is pulled proximately in the longitudinal axis of SCP5, the distal end portion of SCP5 will change from its relaxed, straight state of FIG. 4E left to the compressed, curved, hook-like configuration shown in FIG. 4E right. In addition, the angle e is greater than the angle d of the distal end portion of SCP4 of FIG. 4D.

### Multidirectional Catheter Probe Distal End Portion Deflection

Other example embodiments of this invention feature multidirectional flexion capability wherein the catheter probe distal end portion can be made to deflect in two or more directions. An example of bidirectional deflection is shown in FIG. 5 with steerable catheter probe SCP6 whose distal end portion incorporates a compression coil 30 with one coil, coil 31, of smaller diameter than the other coils and, as necessary for this feature, centered, or closely so, about the central longitudinal axis 35. There are two pull wires 32 and 33 that connect distally to the tightly wound coil at fixation points 34 and 35 respectively. The pull wires are outside of smaller diameter coil 31 but inside all other coils in steerable catheter probe SCP6. It can be clearly understood that applying a longitudinal force to pull wire 32 in a proximal direction will cause the catheter probe distal end portion to deflect “downwards” and relaxing pull wire 32 and applying a similar longitudinal force to pull wire 33 in a proximal direction will cause the catheter probe distal end portion to deflect “upwards,” i.e. in the diametrically opposite direction. This facilitates probe steerability because without this feature, in order to change the direction of deflection of the catheter probe when in use, it must first be rotated through 180 degrees which might cause unintended overall probe movement and loss of trajectory to a tissue target.

Another alternative embodiment of this invention shown in the cross sectional views of coils in FIGS. 6A-E illustrate that, with the exception of FIG. 6E, reducing cross sectional area by changing the shape of one or more coils of a compression coil is a means for accommodating multiple pull wires and thereby obtaining multidirectional flexion capability of a catheter probe distal end portion. In all cases for FIGS. 6A-E, the pull wires are outside the reduced cross sectional area coils. FIG. 6A shows the simple case for a compression coil with circular large diameter coils 40, one or more reduced cross sectional area circular coils 41 that have a concavity in part of their circumference to accommodate a single pull wire 42. In this configuration, a longitudinal proximal pulling force will produce unidirectional deflection of the distal end portion of a catheter probe.

FIG. 6B shows a compression coil with circular large cross sectional area coils 40, one or more reduced cross sectional area circular coils 43 with two diametrically opposite concavities in their circumference to accommodate two pull wires 44 and 45. In this configuration, a longitudinal proximal pulling force on one of the pull wires, e.g. pull wire 44, will produce unidirectional flexion of the distal end portion of a catheter probe in one direction, and a longitudinal proximal pulling force on the other pull wire 45 will produce unidirectional flexion of the distal end portion of a catheter probe in the opposite direction, assuming pull wire 44 is relaxed.

FIG. 6C shows a compression coil with circular large cross sectional area coils 40, one or more reduced cross sectional area three-sided coils 46, in this example shaped as an equilateral triangle, that accommodate exterior to their sides three pull wires 47, 48, and 49. In this configuration, a longitudinal proximal pulling force on one of the pull wires, e.g. 47, will produce unidirectional flexion of the distal end portion of a catheter probe in one direction; a longitudinal proximal pulling force on pull wire 48 will produce unidirectional flexion of the distal end portion of a catheter probe in a direction 120 degrees from that produced by pull wire 47; and a longitudinal proximal pulling force on pull wire 49 will produce unidirectional flexion of the distal end portion of a catheter probe in a direction 240 degrees from that produced by pull wire 47, assuming in all cases the other two pull wires are relaxed.

FIG. 6D shows a compression coil with circular large cross sectional area coils 40, one or more reduced cross sectional area four-sided coils 50, in this example square shaped, that accommodate exterior to their sides four pull wires 51, 52, 53, and 54. In this configuration, a longitudinal proximal pulling force on each the pull wire successively will produce unidirectional flexion of the distal end of a catheter probe in a direction 90 degrees from that produced by the previous pull wire, assuming in all cases that the other three pull wires are relaxed.

FIG. 6E shows a compression coil with circular large cross sectional area coils 40, one or more reduced cross sectional area circular coils 55 coaxial with the large cross sectional area coils 40. Four pull wires 56, 57, 58, and 59 are disposed at 90 degree intervals between circular large cross sectional area coils 40 and reduced cross sectional area circular coils 55. Again, as in the example of FIG. 6D, a longitudinal proximal pulling force on each the pull wire successively will produce unidirectional flexion of the distal end of a catheter probe in a direction 90 degrees from that produced by the previous pull wire.

In the foregoing embodiments of FIGS. 6C-E, where there are three or more reduced cross sectional area coils, the pull wires of two or more of these coils, but not all of them, may be pulled simultaneously to variably control the angle of deflection of the catheter probe distal end. In addition, further and finer control of deflection can be obtained by either changing the catheter probe construction to house more pull wires and/or providing a means for applying variable and independent force of pull on each pull wire.

### Reversibly Controlling Catheter Probe Rigidity

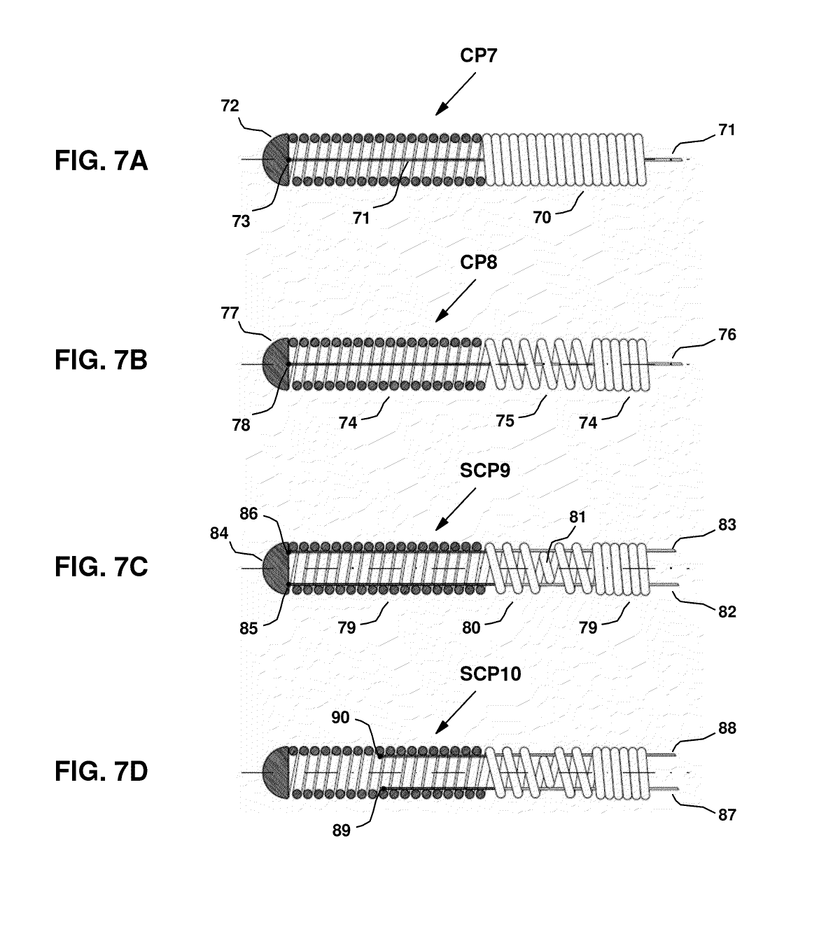

Guidable catheter probes can be required to have conflicting characteristics, rigidity and flexibility; overall rigidity to allow advancement through variably resistant tissue, and flexibility of the catheter probe distal end for maneuverability. This is typically achieved by the initial insertion of a stylet or other stiff member within the catheter lumen, but it is sometimes not possible because of the presence of other components within the lumen such as insulated electrical conductors or multiple pull wires. In addition, the process of stylet removal and hub reconnection can shift the position of the catheter probe. An embodiment of the present invention resolves these problems with catheter probes that during use can be made to vary reversibly from rigid to flexible. Example embodiments of the distal end of such catheter probes are shown in FIGS. 7A-D. In these Figures part of the catheter distal end is shown with the front half of the coil removed to reveal a full section view in order to expose pull wires and pull wire fixation points.

In an example embodiment, FIG. 7A shows the distal end of a catheter probe CP7 constructed of a tightly wound continuous coil 70. Pull wire 71 is positioned along the central longitudinal axis of the catheter probe and is attached to a solid end cap 72 at fixation point 73. In the relaxed state, i.e. no tension applied to pull wire 71, catheter probe CP7 has a flexible body and distal end and can be deflected by dense or inhomogeneous structures as it is advanced through tissue. However with the application of a proximal longitudinal force, catheter probe CP7 becomes increasingly rigid, and as the pulling force intensifies it reaches the point at which it can be advanced without deflection.

In another example embodiment, FIG. 7B shows the distal end of a catheter probe CP8 constructed of a tightly wound continuous coil 74 except for a section where it is loosely wound to function as a compression coil 75. A pull wire 76 is positioned along the central longitudinal axis of the catheter probe and is attached to solid end cap 77 at fixation point 78. In the relaxed state, i.e. no tension applied to pull wire 76, catheter probe CP8 has a flexible body and distal end, but is most flexible where its structure is a compression coil 75 within its distal end. As is the case for catheter probe CP7, catheter probe CP8 will, when a proximal longitudinal force is applied, become increasingly rigid but its distal end but will still be relatively more flexible in the region of compression coil 75, a feature that can beneficially assist steerability to certain tissue regions where passive flexion is desirable. There is a point that with increased pull wire force adjacent coils of compression coil 75 come into contact and rigidity becomes very high and more uniform throughout catheter probe CP8.

In yet another example embodiment, FIG. 7C shows the distal end of a steerable catheter probe SCP9 constructed of a tightly wound continuous coil 79 except for a section where it is loosely wound to function as a compression coil 80. The diameter of all coils in catheter probe SCP9 is equal except for a smaller diameter coil 81 in compression coil 80. There are two pull wires 82 and 83 that connect distally to solid end cap 84 at fixation points 85 and 86 respectively. The pull wires are positioned diametrically opposite each other outside of smaller diameter coil 81 but inside all other coils of steerable catheter probe SCP9. In the relaxed state, i.e. no tension applied to either pull wire 82 or 83, steerable catheter probe SCP9 has a flexible body and distal end and can be deflected by dense or inhomogeneous structures as it is advanced through tissue. However with the application of a proximal longitudinal force to either pull wire 82 or 83, catheter probe SCP9 becomes increasingly rigid and the probe distal end flexes in the manner describe in the embodiment of FIG. 5. Alternatively, if both pull wires are pulled equally and simultaneously, steerable catheter probe SCP9 becomes rigid and remains straight which is another feature of the construction of this embodiment.

In a last example embodiment, FIG. 7D shows the distal end of steerable catheter probe SCP10 which is identical to SCP9 except its pull wires 87 and 88 are connected more proximally to tightly wound coils of the distal end at fixation points 89 and 90 respectively. Application of a proximal longitudinal force to either pull wire 87 or 88 produces similar increases in rigidity and distal tip flexion as described for catheter probe SCP9 except the portion of the coil beyond fixation points 89 and 90 remains flexible at all times.

A feature of all example embodiments of FIGS. 7A-D is that once a catheter probe reaches a selected position the force on its pull wires can be released with consequent return of the catheter probe to its relaxed state of flexibility, as may be desired for certain procedures or for chronic implantation of a catheter probe.

### INCORPORATION BY REFERENCE

This application claims the benefits of U.S. patent application Ser. No. 13/188,101 filed Jul. 21, 2011, the entire contents of which are hereby incorporated by reference. Specifically, a multiplicity of functions can be incorporated within the embodiments of the present invention:
(i) A first function is as a catheter for the injection of fluids into body spaces and tissues for diagnostic or therapeutic purposes. Fluid injected into a proximal hub of the catheter probe exits through the loosely wound coils of the catheter distal end.
(ii) A second function is as a probe for the application of an electrical stimulus to targeted tissue that is in contact with or close to the catheter distal end. The insulated tightly wound stainless steel coil of the tubular catheter body serves as a conductive pathway to the uninsulated catheter distal end which acts as an electrode. A stimulus response can be used to confirm the accuracy of the placement of the catheter distal end before therapeutic procedures are initiated, or stimulus current can be used for short or long term therapeutic benefit such as the alleviation of chronic spinal pain.
(iii) A third function is as a probe for the application of ablation energy such as continuous or pulsed radiofrequency (RF) energy to a targeted tissue. In the same manner as in (ii) above, the uninsulated catheter probe distal end acts as an electrode, in this case for RF energy application to a target tissue which is in contact with or in close proximity to the catheter probe distal end.
(iv) A fourth function is as a means for measuring electrical impedance of tissue or fluids at the catheter distal end when the catheter probe is connected to an instrument with an impedance measurement module. Impedance values can be used, for example, as a confirmation of the location of the catheter distal end or for assessing the effectiveness of an RF ablation procedure by change in impedance. For this function, the catheter distal end again acts as an electrode.
(v) A fifth function is a means for monitoring tissue temperature. A very small thermocouple sensor is positioned within the lumen of the catheter distal end to measure the change in tissue temperature related to the application of, for example, RF ablation energy. The thermocouple sensor is connected to a temperature measuring instrument via one or two electrical leads from the sensor.

The foregoing provides a detailed description of exemplary embodiments of the present invention. Although embodiments of steerable multifunction catheter probes with high guidability and reversible rigidity for diagnostic and/or therapeutic purposes have been described with reference to preferred embodiments and examples thereof, other embodiments and examples may perform similar functions and/or achieve similar results. All such equivalent embodiments and examples are within the spirit and scope of the present invention and are intended to be covered by the following claims.

That which is claimed is:

1. A catheter probe, comprising:
a catheter body having a body portion adapted for being connected to a proximal hub and a distal end portion connected to the body portion, wherein the catheter body defines a lumen;
the distal end portion having a compressible segment and a non-compressible segment, the compressible segment having a longitudinal centerline; and
a pull member attached to the distal end portion and adapted for applying a proximally directed force to the distal end portion whereby at least a portion of said compressible segment is compressed.

2. The catheter probe of claim 1, wherein the pull member is attached to the distal end portion at a location coaxial with the longitudinal centerline of the compressible segment whereby application of the proximal force increases rigidity of the distal end portion.

3. The catheter probe of claim 1, wherein the pull member is attached to the distal end portion at a location offset a distance from being coaxial with the longitudinal centerline of the compressible segment whereby application of the proximal force causes flexion of the distal end portion relative to the longitudinal centerline.

4. The catheter probe of claim 1, wherein the pull member is attached to the distal end portion at a location distal of the compressible segment.

5. The catheter probe of claim 1, wherein the pull member is attached to the distal end portion at the compressible segment.

6. The catheter probe of claim 1, wherein the distal end portion is a coil.

7. The catheter probe of claim 1, wherein the compressible segment is a coil having open adjacent loops.

8. The catheter probe of claim 7, wherein the non-compressible segment is a coil having engaged adjacent loops.

9. The catheter probe of claim 7, wherein the compressible segment coil has a first coil of a first diameter adjacent a second coil of a second diameter, wherein the first coil diameter is larger than the second coil diameter.

10. The catheter probe of claim 1 further comprising a second pull member attached to the distal end portion and adapted for applying a proximally directed force to the distal end portion whereby at least a portion of said first compressible segment is compressed.

11. The catheter probe of claim 10, wherein the first and second pull members, individually or cooperatively, are capable of applying proximally directed force to increase rigidity and/or allow multidirectional flexion of the distal end portion relative to the longitudinal centerline of the first compressive segment.

12. A catheter probe, comprising:
a catheter body having a body portion adapted for being connected to a proximal hub and a distal end portion connected to the body portion, wherein the catheter body defines a lumen;
the distal end portion having a first compressible segment, second compressible segment disposed distally from the first compressible segment, and a non-compressible segment disposed between the first and second compressible segments, the compressible segments each having a longitudinal centerline; and
a first pull member attached to the distal end portion and adapted for applying a proximally directed force to the distal end portion whereby at least a portion of said first compressible segment is compressed.

13. The catheter probe of claim 12, wherein the pull member is attached to the distal end portion at a location distal to the second compressible segment and offset a distance from being coaxial the longitudinal centerline of either compressible segment whereby application of the proximal force causes flexion of the distal end portion relative to the longitudinal centerline of the first compressible segment.

14. The catheter probe of claim 13, wherein distal end portion is capable of flexion of between 45 degrees and 90 degrees relative to the longitudinal centerline of the first compressible segment.

15. The catheter probe of claim 13, wherein distal end portion is capable of flexion of between 90 degrees and 180 degrees relative to the longitudinal centerline of the first compressible segment.

16. The catheter probe of claim 12, wherein the first compressible segment coil has a first coil of a first diameter adjacent a second coil of a second diameter, wherein the first coil diameter is larger than the second coil diameter.

17. The catheter probe of claim 16, wherein the second compressible segment coil has a first coil of a first diameter adjacent a second coil of a second diameter, wherein the first coil diameter is larger than the second coil diameter.

18. The catheter probe of claim 13, further comprising a second pull member attached to the distal end portion and adapted for applying a proximally directed force to the distal end portion whereby at least a portion of said first compressible segment is compressed.

19. The catheter probe of claim 18, wherein the first and second pull members, individually or cooperatively, are capable of applying proximally directed force to increase rigidity of the distal end portion.

20. The catheter probe of claim 18, wherein the first and second pull members, individually or cooperatively, are capable of applying proximally directed force to allow multidirectional flexion of the distal end portion relative to the longitudinal centerline of the first compressive segment.
